(19) United States
(12) Patent Application Publication
(10) Pub. No.: US 2022/0001054 A1
(43) Pub. Date: Jan. 6, 2022

(54) DISINFECTING LIGHT FIXTURE HAVING DETECTION DEVICE

(71) Applicants: Pu-Dan Limited Corp., New Taipei City (TW); National Taipei University of Technology, Taipei (TW)

(72) Inventors: Wei-Chih HUANG, New Taipei City (TW); Yuh-Shyan HWANG, Taipei (TW); Tung-Jing FANG, Taipei (TW); Chun-Young CHANG, Taipei (TW); Chih-Wen WEN, Taipei (TW)

(21) Appl. No.: 17/367,236

(22) Filed: Jul. 2, 2021

(30) Foreign Application Priority Data
Jul. 6, 2020 (TW) 109122831

(51) Int. Cl. A61L 2/10; G01S 13/04

(52) U.S. Cl. CPC A61L 2/10; G01S 13/04; A61L 2202/25; A61L 2202/14; A61L 2202/11

(57) ABSTRACT

A disinfecting light fixture including a disinfecting lamp and a detection device is provided. The disinfecting lamp is disposed in an indoor space to emit disinfection light in the indoor space. The detection device is coupled to the disinfecting lamp to turn on or off the disinfecting lamp and includes an emitter, an antenna, a receiver, a signal processor and an actuator. The detection device is configured to detect the condition of the indoor space. In response to a human in the indoor space, the detection device turns off the disinfecting lamp. In response to there being no humans in the indoor space, the detection device turns on the disinfecting lamp.

### CROSS REFERENCE TO RELATED APPLICATIONS

This application claims priority of Taiwan Patent Application No. 109122831, filed on Jul. 6, 2020, the entirety of which is incorporated by reference herein.

### BACKGROUND OF THE INVENTION

### Field of the Invention

The invention relates to a disinfecting light fixture, and more particularly to a disinfecting light fixture that comprises a detection device detecting indoor space conditions and is turned on or off according to the detection result generated by the detection device.

### Description of the Related Art

Viruses are inevitable in the daily life of human beings. Meeting rooms and elevators that people must use are all confined spaces. Viruses spread very easily in these and other confined spaces. Therefore, how to eliminate viruses in confined spaces through modern technology is very important. For example, one conventional method uses detergent or alcohol to eliminate viruses in elevators and meeting rooms. If it is not wiped clean after use, viruses may remain on desktops and elevator buttons, or even in the air, causing subsequent users to become infected by the viruses.

In order to solve the above-mentioned problems, the industry has used ultraviolet rays to disinfect and sterilize indoor environments. According to research materials, ultraviolet rays mainly damage the DNA or RNA in microbial cells and cause the death of microbes, thereby achieving the effect of disinfection and sterilization.

However, ultraviolet rays have a strong lethality to microorganisms, and can also cause harm to the human body, such as skin allergies, skin tumors, retinopathy, cataracts, etc. Therefore, people cannot enter a room in which ultraviolet lamps are being used for disinfection and sterilization, which means that users frequently need to turn the ultraviolet lamps on and off.

Therefore, how to solve the above-mentioned problems and deficiencies is the path that the inventor of the present invention and related manufacturers engaged in this industry are eager to study and improve.

### BRIEF SUMMARY OF THE INVENTION

In view of the above-mentioned deficiencies, the inventors of the present invention have collected relevant information, evaluated it, and considered input from many parties, and based on their years of experience in this industry, and after continuous trials and modifications, have designed this invention patent.

The purpose of the present invention is to provide a disinfecting light fixture that comprises a detection device detecting indoor space condition and is turned on or off according to the detection result.

In order to achieve the above or other objectives, the present invention provides a disinfecting light fixture that comprises a disinfecting lamp disposed in an indoor space to emit disinfection light in the indoor space, and a detection device coupled to the disinfecting lamp to turn on or off the disinfecting lamp and comprising: an emitter configured to generate a radio frequency electrical signal; an antenna coupled to the emitter to convert the radio frequency electrical signal into an electromagnetic wave and emitting the electromagnetic wave, wherein the wavelength of the electromagnetic wave is between 1 millimeter and 15 millimeters, and the antenna is configured to receive a reflected electromagnetic wave; a receiver coupled to the antenna to receive a reflected electromagnetic wave and converting the reflected electromagnetic wave into a low-frequency signal; a signal processor coupled to the receiver to receive the low-frequency signal and processing the low-frequency signal to generate at least one driving signal; and an actuator coupled to the signal processor to receive the driving signal and turning on or off the disinfecting lamp according to the driving signal.

In one embodiment, the disinfecting lamp is an ultraviolet lamp.

In one embodiment, the disinfecting light fixture further comprises a power source coupled to the disinfecting lamp and the detection device to supply power.

In one embodiment, the signal processor comprises a digital signal processor and a microcontroller unit.

In one embodiment, the disinfecting light fixture further comprises a monitoring center coupled to the actuator and the disinfecting lamp to receive the driving signal and turning on or off the disinfecting lamp according to the driving signal.

In one embodiment, the disinfecting lamp comprises a manual switch configured to turn on or off the disinfecting lamp.

### BRIEF DESCRIPTION OF THE DRAWINGS

The invention can be more fully understood by referring to the following detailed description and examples with references made to the accompanying drawings, wherein:

FIG. 1 is a block diagram of an exemplary embodiment of a disinfecting light fixture with a detection device according to various aspects of the present disclosure.

FIG. 2 is a schematic diagram of an exemplary embodiment of the disinfecting light fixture with a detection device according to various aspects of the present disclosure.

FIG. 3 is a schematic diagram of another exemplary embodiment of the disinfecting light fixture with a detection device according to various aspects of the present disclosure.

FIG. 4 is a schematic diagram of another exemplary embodiment of the disinfecting light fixture with a detection device according to various aspects of the present disclosure.

### DETAILED DESCRIPTION OF THE INVENTION

In order to achieve the above-mentioned purposes and effects, the technical means and structure adopted by the present invention are illustrated in a schematic manner and in conjunction with the preferred embodiments of the present invention, and its features and functions are described in detail as follows, for the benefit of a complete understanding.

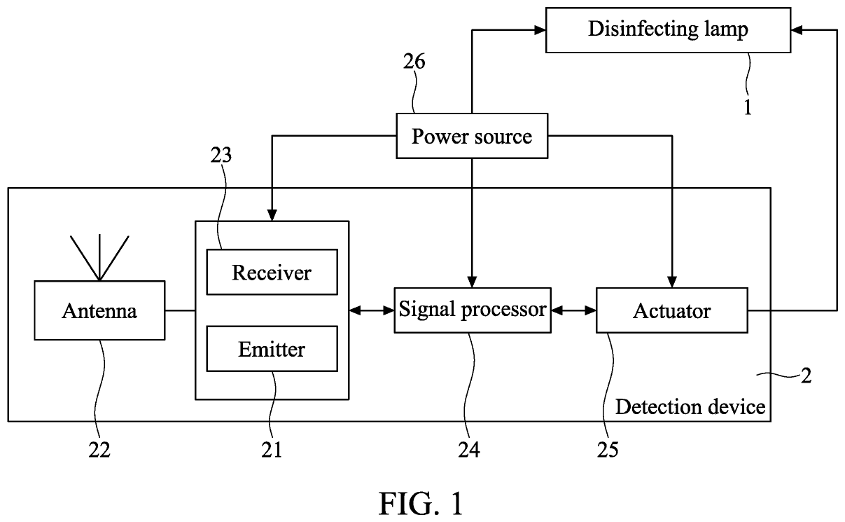

FIG. 1 is a block diagram of an exemplary embodiment of a disinfecting light fixture with a detection device according to various aspects of the present disclosure. As shown in FIG. 1, the disinfecting light fixture comprises a disinfecting lamp 1 and a detection device 2.

The disinfecting lamp 1 is disposed in an indoor space to emit disinfection light in the indoor space. In one embodiment, the disinfecting lamp 1 is an ultraviolet lamp. However, other lamps that have similar effects and can also achieve disinfection and sterilization functions are also feasible solutions. Additionally, the disinfecting lamp 1 may be comprise a manual switch configured to turn on or off the disinfecting lamp 1.

The detection device 2 is coupled to the disinfecting lamp 1 to turn on or off the disinfecting lamp 1. The detection device 2 comprises an emitter 21, an antenna 22, a receiver 23, a signal processor 24 and an actuator 25.

The emitter 21 is configured to generate a radio frequency electrical signal.

The antenna 22 is coupled to the emitter 21 to convert the radio frequency electrical signal into an electromagnetic wave and emits the electromagnetic wave. The wavelength of the electromagnetic wave is between 1 millimeter and 15 millimeters. Furthermore, the antenna 22 is configured to receive a reflected electromagnetic wave.

The receiver 23 is coupled to the antenna 22 to receive the reflected electromagnetic wave. The receiver 23 converts the reflected electromagnetic wave into a low-frequency signal.

The signal processor 24 is coupled to the receiver 23 to receive the low-frequency signal. The signal processor 24 processes the low-frequency signal to generate at least one driving signal. In one embodiment, the signal processor 24 comprises a digital signal processor (DSP) and a microcontroller unit (MCU), but the disclosure is not limited thereto.

The actuator 25 is coupled to the signal processor 24 to receive the driving signal, and turns on or off the disinfecting lamp 1 according to the driving signal.

Additionally, the disinfecting light fixture further comprises a power source 26. The power source 26 is coupled to the disinfecting lamp 1 and the detection device 2 to supply power.

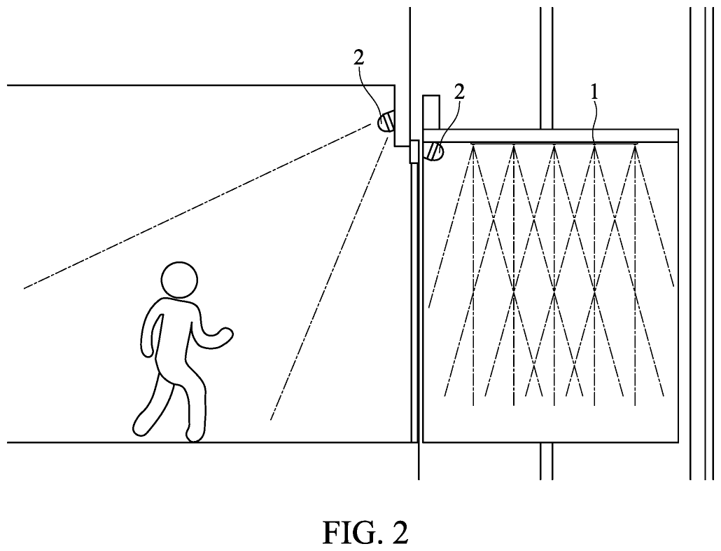

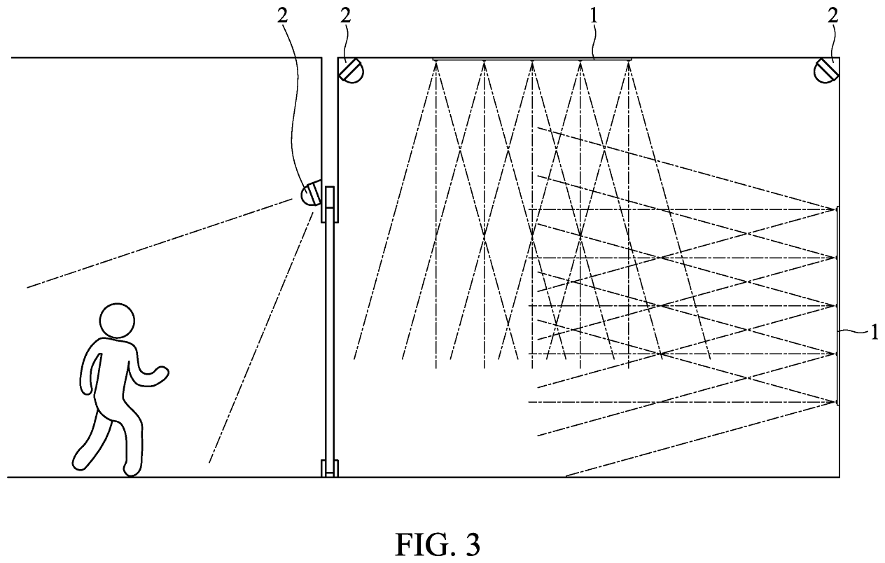

With the above-mentioned structure and composition design, the operation of the present invention is now described as follows: please refer to FIGS. 2 and 3 simultaneously. FIG. 2 is a schematic diagram of an exemplary embodiment of the disinfecting light fixture with a detection device according to various aspects of the present disclosure. FIG. 3 is a schematic diagram of another exemplary embodiment of the disinfecting light fixture with a detection device according to various aspects of the present disclosure. As shown in FIGS. 2 and 3, the disinfecting lamp 1 may be disposed in an indoor space to emit disinfection light in the indoor space. For example, the disinfecting lamp 1 is disposed on the ceiling or the wall. The detection device 2 may be also disposed in the same indoor space. In one embodiment, the detection device 2 is disposed in the doorway of the indoor space. Therefore, the detection device 2 is utilized to detect the indoor space condition. If someone is in the indoor space or is about to enter the indoor space, the ultraviolet lamp for disinfection and sterilization may be turned off. On the contrary, if there is no one in the indoor space, or no one is about to enter the indoor space, the ultraviolet lamp for disinfection and sterilization may be turned on.

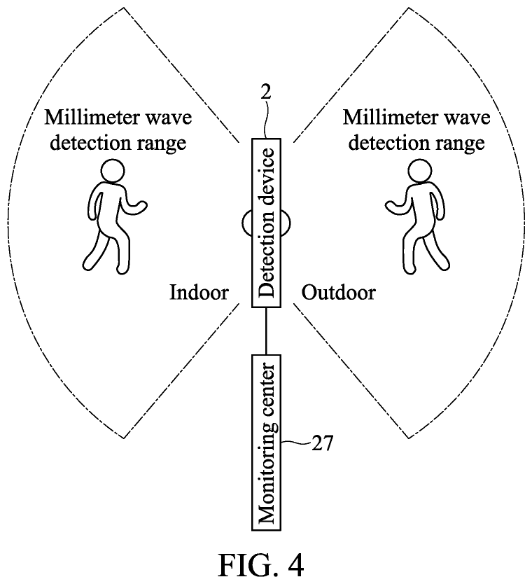

FIG. 4 is a schematic diagram of another exemplary embodiment of the disinfecting light fixture with a detection device according to various aspects of the present disclosure. As shown in FIG. 4, when the detection device 2 detects a person who is about to enter the indoor space, the actuator 25 of the detection device 2 may turn off the disinfecting lamp 1. In other embodiments, when the detection device 2 does not detect a person who is about to enter the indoor space, the actuator 25 of the detection device 2 may turn on the disinfecting lamp 1 to emit disinfection light in the indoor space. Furthermore, when the disinfecting lamp 1 is turned on, the disinfecting lamp 1 can also emit warning messages such as light or sound to inform that it is currently being disinfected.

The disinfecting light fixture further comprises a monitoring center 27. The monitoring center 27 is coupled to the actuator 25 and the disinfecting lamp 1 to receive the driving signal and turns on or off the disinfecting lamp 1 according to the driving signal. The monitoring center 27 may obtain whether there are people in the indoor space according to the driving signal. Therefore, the operator of the monitoring center 27 can control the disinfecting lamp 1 to turn off or on according to the actual condition.

Please refer to all the drawings, when the present invention is used and compared with the prior art, the present invention has the following advantages. The disinfecting light fixture utilizes the detection device 2 to detect the indoor space condition to turn on or off the disinfecting lamp 1 according to the detection result.

The above description is only an embodiment of the present invention, and is not intended to limit the scope of the patent of the present invention. Therefore, all simple modifications and equivalent structural changes made by using the description and schematic content of the present invention should be included in the scope of the patent of the present invention.

What is claimed is:

1. A disinfecting light fixture comprising:
a disinfecting lamp disposed in an indoor space to emit disinfection light in the indoor space; and
a detection device coupled to the disinfecting lamp to turn on or off the disinfecting lamp and comprising:
an emitter configured to generate a radio frequency electrical signal;
an antenna coupled to the emitter to convert the radio frequency electrical signal into an electromagnetic wave and emitting the electromagnetic wave, wherein the wavelength of the electromagnetic wave is between 1 millimeter and 15 millimeters, and the antenna is configured to receive a reflected electromagnetic wave;
a receiver coupled to the antenna to receive a reflected electromagnetic wave and converting the reflected electromagnetic wave into a low-frequency signal;
a signal processor coupled to the receiver to receive the low-frequency signal and processing the low-frequency signal to generate at least one driving signal; and
an actuator coupled to the signal processor to receive the driving signal and turning on or off the disinfecting lamp according to the driving signal.

2. The disinfecting light fixture as claimed in claim 1, wherein the disinfecting lamp is an ultraviolet lamp.

3. The disinfecting light fixture as claimed in claim 1, further comprising:
a power source coupled to the disinfecting lamp and the detection device to supply power.

4. The disinfecting light fixture as claimed in claim 1, wherein the signal processor comprises a digital signal processor and a microcontroller unit.

5. The disinfecting light fixture as claimed in claim 1, further comprising:
a monitoring center coupled to the actuator and the disinfecting lamp to receive the driving signal and turning on or off the disinfecting lamp according to the driving signal.

6. The disinfecting light fixture as claimed in claim 1, wherein the disinfecting lamp comprises a manual switch configured to turn on or off the disinfecting lamp.
